(19) United States
(12) Patent Application Publication
(10) Pub. No.: US 2006/0293657 A1
(43) Pub. Date: Dec. 28, 2006

(54) Damping element

(76) Inventor: Stephan Hartmann, Solothurn (CH)

Correspondence Address: JONES DAY, 222 EAST 41ST STREET, NEW YORK, NY, 10017-6702, US

(21) Appl. No.: 11/393,484

(22) Filed: Mar. 29, 2006

Related U.S. Application Data
(63) Continuation of application No. PCT/CH03/00648, filed on Sep. 29, 2003.

(51) Int. Cl. A61B 17/56

(52) U.S. Cl. 606/60

(57) ABSTRACT

A damping element for the dynamic stabilization of two bones, particularly of two adjacent bodies of the vertebra, has a central axis, a first end intersecting the central axis, a second end intersecting the central axis, and a spring element between the two ends and coaxial with the central axis. The damping element also has a ball-joint connection at at least one end that is concentric with the central axis. The ball-joint connection operative to receive and releasably lock a rod-shaped longitudinal support therein.

### CROSS REFERENCE TO RELATED APPLICATION

This is a continuation of International Patent Application No. PCT/CH2003/00648, filed Sep. 29, 2003, the entire contents of which are incorporated herein by reference thereto.

### TECHNICAL FIELD OF THE INVENTION

The invention is directed to a damping element having a spring element and at least one ball-joint connection for receiving a rod-shaped longitudinal support.

### BACKGROUND OF THE INVENTION

Damping elements for the dynamic stabilization of two adjacent bodies of the vertebra are known. For example, one known damping element comprises a coaxial damping body with a spherically convex axially protruding connecting part at each axial end that can be secured to two respective pedicle screws. By virtue of the spherical joint between the two connecting parts and the heads of the pedicle screws, the damping element can be connected to the pedicle screws with varying angles between the longitudinal axes of the pedicle screws and the central axis of the damping element. A disadvantage of this known damping element is that due to the geometry of the damping element, the distance between the pedicle screws is predetermined.

### SUMMARY OF THE INVENTION

The object of the invention is to produce a damping element that can polyaxially pivot about at least one of its ends and is axially, telescopingly connected to a longitudinal support.

The advantages achieved by the invention of a damping element having a ball joint connection at at least one end of the damping element include:
in the unlocked state of the ball joint, the damping element can be polyaxially pivotably connected to a rod-shaped longitudinal support of a device to stabilize bodies of the vertebra. For this reason, during the implanting of a longitudinal support, no longitudinal support needs to be bent within a vertebra-stabilizing device, and
the damping element can be axially telescopingly connected to a longitudinal support of a vertebra-stabilizing device.

In a preferred embodiment, the ball joint comprises a spherically convex, radially compressible clamping body with a diametral central bore having a bore axis. When the clamping body is compressed, a rod-shaped longitudinal support introduced into the central bore is locked in the central bore relative to the clamping body.

The ball joint allows preferably a rotation of the clamping body by an angle α, in the range from 0° to ±25 °, measured between the bore axis of the central bore in the clamping body and the central axis of the spring element of the damping element. This advantageously allows a rod-shaped longitudinal support introduced into the central bore of the clamping body to pivot relative to the spring element and, consequently, the rod-shaped longitudinal support does not have to be bent.

The ball joint preferably comprises two axially separated bearing shells, accommodating at least partially the clamping body, so that when the bearing shells are compressed, the clamping body is equally compressed and thus the ball joint can be rigidly locked.

In another embodiment, the bearing shells can be pressed against the clamping body by tightening means, whereby preferably the spring element has at its first end a coaxial spigot with a thread, and the first bearing shell is integrated axially at the end in the spigot in such a manner that the bearing shell converges towards the second end of the spring element. The tightening means is preferably constructed as a nut that can be screwed onto the thread of the spigot. The second bearing shell is preferably concentrically integrated in the bore of the nut.

In a further embodiment, the nut comprises a coaxial bore with at least two axially adjacent longitudinal sections. The outer longitudinal section facing the spring element has an inside thread that is complementary to the thread of the spigot. In the adjacent longitudinal section, the second bearing shell is integrated in such a manner that it expands towards the outer longitudinal section.

The tightening means is bored through to enable a rod-shaped longitudinal support to pass there through.

In yet another further embodiment, the clamping body has a slot that is parallel to the bore axis, the slot penetrating the wall of the clamping body from its external wall up to the central bore.

In another embodiment, the damping element comprises a rod-shaped connecting part that is coaxial at its ends. The connecting part can be joined with a further part within an osteosynthetic stabilizing device.

In yet another embodiment, the damping element additionally comprises a rod-shaped longitudinal support that can be introduced into the central bore of the clamping body and can be releasably fixed in the clamping body.

### BRIEF DESCRIPTION OF THE DRAWINGS

The detailed description will be better understood in conjunction with the accompanying drawings, in which like reference characters represent like elements as follows:

FIG. 1 is a longitudinal cross-sectional view of an embodiment of a damping element; and

FIG. 2 is an enlarged, more detailed cross-sectional view of section A in FIG. 1.

### DETAILED DESCRIPTION OF THE INVENTION

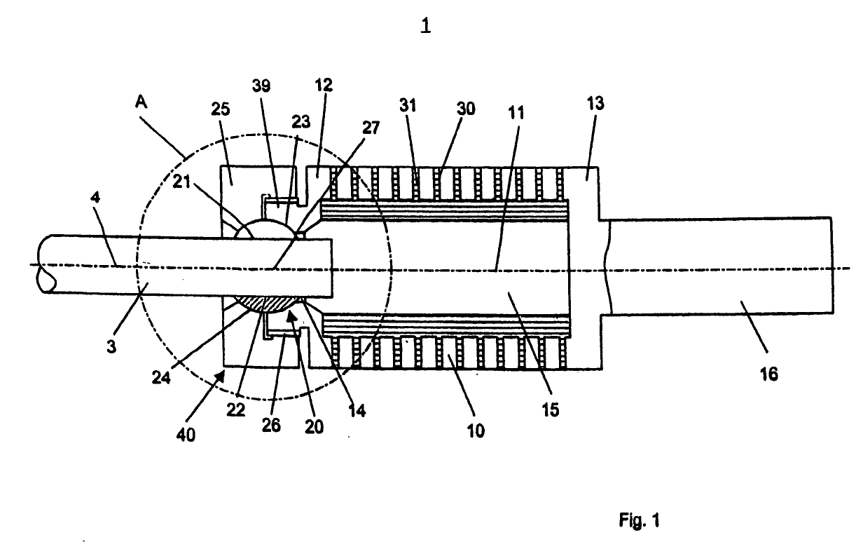

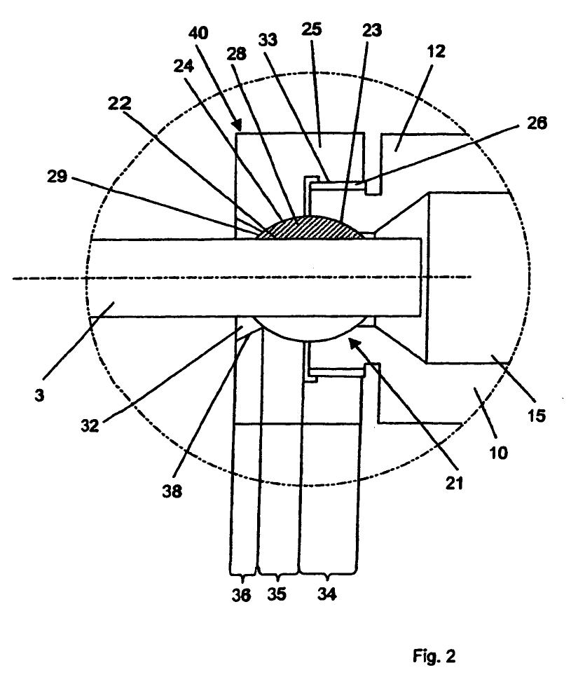

FIGS. 1 and 2 illustrate an embodiment that comprises a hollow cylindrical damping element 1 with a central axis 11 and a releasably lockable ball joint 20 for a polyaxial connection of the damping element 1 with a rod-shaped longitudinal support 3 having a longitudinal axis 4. In addition to the ball joint 20, the damping element 1 includes a spring element 10 that in the embodiment illustrated is made from a metal helical spring and a plastic part 31 which penetrates into a gap 30 between the coils of the spring and reduces the diameter of the hollow space 15. The ball joint 20 is provided on the first end 12 of the spring element 10, whereas on the second, axially opposed end 13 of the spring element 10, a coaxial rod-shaped connecting part 16 is provided. Connecting part 16 is suitable to be connected to a further part (not illustrated) of a vertebra-stabilizing device.

The ball joint 20 comprises in this case a spherically convex clamping body 21 with a central bore 22 having a bore axis 27 and two concave bearing shells 23, 24, which are complementary to the clamping body 21. The first bearing shell 23 is integrated in the threaded spigot 39 concentrically with the central axis 11 on the first end 12 of spring element 10 in such a manner that it converges towards the hollow space 15 in damping element 1. The threaded spigot 39 has a bore 14 coaxially with the central axis 11 that terminates in the hollow space 15 such that the bore 14 is suitable to accommodate a rod-shaped longitudinal support 3, which is guided through the central bore 22 in the clamping body 21. The second bearing shell 24 is integrated in a nut 25 that can be screwed on the threaded spigot 39 via the thread 26. The hollow space 15 is closed at the second end 13 of the spring element 10. To join the second end 13 of the spring element 10 with a further part, for example the head of a pedicle screw or pedicle hook (not illustrated), a rod-shaped connecting part 16 coaxial with the central axis 11 is provided on the second end 13 of the spring element 10.

As shown in FIG. 2, the clamping body 21 is provided with slots 28 which are parallel to the bore axis 27, the slots penetrating the clamping body 21 from the external wall 29 of the clamping body 21 up to the central bore 22. When the nut 25 is tightened, the clamping body 21, which is provided between the bearing shells 23, 24, is clamped between the bearing shells 23, 24 and simultaneously radially compressed towards the bore axis 27 of the central bore 22, so that the longitudinal support 3, introduced into the central bore 22, will be locked.

The nut 25 has a bore 32 that is coaxial with the central axis 11. Bore 32 having a plurality of axially adjacent longitudinal sections 34, 35, 36 with various geometries. The longitudinal section 34, adjacent to the first end 12 of damping element 1, is provided with an inside thread 33 that is complementary to the thread 26 on the first end 12 of spring element 10. The middle longitudinal section 35 includes the second bearing shell 24, which is also bored through. The externally situated longitudinal section 36 has a tapered construction. At the same time, the second bearing shell 24 is arranged such that it converges towards the externally situated longitudinal section 36. The taper 38 in the externally situated longitudinal section 36 expands towards the external face 37 of the nut 25, so that a rod-shaped longitudinal support 3 can be pivotally accommodated in the ball joint 20.

1. A damping element for the dynamic stabilization of two bones, the damping element comprising:
a spring element having a first and second ends and a central axis intersecting the two ends; and
a ball joint concentric with the central axis and located at the first end, the ball joint having tightening means to releasably lock therein a rod-shaped longitudinal support.

2. The damping element of claim 1 wherein the ball joint comprises a spherically convex clamping body having a diametral central bore, the central bore having a bore axis.

3. The damping element of claim 2 wherein the ball joint allows rotation of the clamping body by an angle a ranging from 0° to ±25°, angle α measured between the bore axis and the central axis.

4. The damping element of claim 2 wherein the ball joint comprises two axially separated bearing shells that accommodate at least partially the clamping body.

5. The damping element of claim 4, wherein the bearing shells can be pressed axially against the clamping body by the tightening means.

6. The damping element of claim 4 wherein the spring element has at the first end a coaxial spigot with a thread, and wherein an end of the first bearing shell is integrated axially in the spigot such that the bearing shell converges towards the second end.

7. The damping element of claim 6 wherein the tightening means comprises a nut that can be screwed onto the thread of the spigot and that the second bearing shell is concentrically joined to the nut.

8. The damping element of claim 7 wherein the nut comprises a coaxial bore with at least first and second axially adjacent longitudinal sections, the first longitudinal section being closest to the spring element and having an inside thread complementary to the spigot thread, and the second longitudinal section having the second bearing shell integrated therewith such that the second bearing shell expands towards the first longitudinal section.

9. The damping element of claim 1 wherein the tightening means is bored through coaxially.

10. The damping element of claim 2 wherein the clamping body has an external wall and a slot parallel to the bore axis that penetrates the clamping body from the external wall.

11. The damping element of claim 1 further comprising a rod-shaped connecting part coaxially joined at the second end.

12. The damping element of claim 1 further comprising a rod-shaped longitudinal support inserted into the central bore and releasably fixed in the ball joint.
